(19) United States
(12) Patent Application Publication
(10) Pub. No.: US 2012/0029048 A1
(43) Pub. Date: Feb. 2, 2012

(54) PHENYLALKYL N-HYDROXYUREAS FOR TREATING LEUKOTRIENE RELATED PATHOLOGIES

(75) Inventors: Rebecca Taub, Villanova, PA (US); Tilmann Brotz, Berkeley, CA (US); John Franc, Newtown, PA (US); Larry Cohen, Oakland, CA (US); Hemantkumar H. Patel, Bodakdev Ahmedabad (IN); Sanjay R. Chemburkar, Gurnee, IL (US); David P. Sawick, Gurnee, IL (US)

(73) Assignee: VIA Pharmaceuticals, Inc., Fort Washington, PA (US)

(21) Appl. No.: 13/025,106

(22) Filed: Feb. 10, 2011

Related U.S. Application Data
(60) Provisional application No. 61/369,462, filed on Jul. 30, 2010.
(60) Provisional application No. 61/438,798, filed on Feb. 2, 2011.

(51) Int. Cl. A61K 31/381; A61P 9/10; A61P 25/28; A61P 19/02; A61P 11/06; A61P 1/00; C07D 333/20; A61P 11/00

(52) U.S. Cl. 514/438; 549/75

(57) ABSTRACT

The method of treating patients by administering N-[3-[5-[(4-fluorophenyl)methyl]-2-thienyl]-1-methyl-2-propynyl]-N-hydroxyurea for treatment of leukotriene related pathologies and compositions for this use.

### CROSS REFERENCE TO RELATED APPLICATIONS

This application claims the benefit of priority under 35 U.S.C. §119 to U.S. Provisional Patent Application Ser. No. 61/369,462, filed on Jul. 30, 2010, and U.S. Provisional Patent Application Ser. No. 61/438,798, filed on Feb. 2, 2011, the entire disclosures of which are incorporated by reference herein.

### FIELD OF THE INVENTION

This invention is in the field of preventing and treating atherosclerotic plaque, cardiovascular diseases, and other inflammatory diseases including chronic obstructive pulmonary disease (COPD), ocular inflammatory diseases, asthma, allergic rhinitis, rheumatoid arthritis, cancers including leukemias and lymphomas, psoriasis, adult respiratory distress syndrome, inflammatory bowel disease, endotoxin shock syndrome, ischemia induced myocardial injury, and central nervous system pathology resulting from formation of leukotrienes following stroke or subarachnoid hemorrhage.

### BACKGROUND OF THE INVENTION

The build up of fat-laden deposits on vessel walls as atherosclerotic plaque causes progressive narrowing in the vessel, such as in a carotid or coronary artery. Eventually, lumen or blood flow within the vessel is reduced to such a level that tissue, such as a heart muscle or brain tissue, is starved of oxygen-carrying blood which produces cardiovascular disease resulting in a heart attack, stroke or peripheral ischemia (reduced blood flow to feet or legs). In this process, low-density lipoproteins (LDLs) and immune system cells accumulate in the vessel wall and attract immune system cells into the vessel wall as well. Immune system cells ingest the modified LDLs, giving rise to fatty droplets, which constitute a lipid core of the plaque. The immune system cells secrete enzymes that degrade collagen of the fibrous cap of the plaque and prevent the development of new collagen fibers to repair the cap damage. The weakening of the cap may result in plaque rupture during which the blood of the lumen intermingles with the lipid core, rich in proteins that foster blood coagulation. As a result, a clot forms and the vessel may be occluded. This sudden occlusion of the blood vessel reduces or stops blood flow to the tissue, which results in death of heart muscle or brain tissue due to lack of oxygen-carrying blood resulting in heart attack or stroke. These acute events relating to plaque rupture are the major causes of morbidity and mortality in patients suffering from cardiovascular diseases.

Plaque composition in arteries is indicative of the risk of acute coronary syndromes. Soft plaque includes a high lipid concentration, a thin fibrous cap and inflammatory cells. Plaques with these characteristics are at increased risk for rupture and the associated acute events.

In the past, the build-up of atherosclerotic plaque has been treated by the use of anti-hypercholesterolemia and anti-hyperlipidemia agents to prevent the build-up of blood cholesterol. While these agents have been successful in reducing the levels of cholesterol and lipids in the blood, they do not directly treat the underlying causes of plaque rupture which lead to a risk of acute events. Therefore patients treated with existing agents may still be prone to plaque rupture and acute events.

In addition to cardiovascular diseases, leukotriene inhibitors have potential for efficacy in a large number of diseases. Leukotrienes have a multitude of biologic actions and have been suggested as factors in numerous disease processes involving inflammation including chronic obstructive pulmonary disease (COPD), ocular inflammatory diseases, asthma, allergic rhinitis, rheumatoid arthritis, cancers including leukemias and lymphomas, psoriasis, adult respiratory distress syndrome, inflammatory bowel disease, endotoxin shock syndrome, ischemia induced myocardial injury, and central nervous system pathology resulting from formation of leukotrienes following stroke or subarachnoid hemorrhage.

However, there is a lack of effective agents that act as leukotriene inhibitors.

Therefore, it is long to be desired to provide an agent which will be effective preventing and treating cardiovascular diseases caused by atherosclerotic plaque through stabilizing the plaque and as well as preventing the formation of atherosclerotic plaque thereby reducing the risk of plaque rupture and acute events as well as effective leukotriene inhibitors.

### BRIEF DESCRIPTION OF THE DRAWINGS

FIG. 1 is a line graph showing mean leukotriene B4 (LTB4) production with increasing doses of N-[3-[5-[(4-fluorophenyl)methyl]-2-thienyl]-1-methyl-2-propynyl]-N-hydroxyurea over 12 weeks.

FIG. 2 is a line graph showing mean leukotriene E4 (LTE4) production with increasing doses of N-[3-[5-[(4-fluorophenyl)methyl]-2-thienyl]-1-methyl-2-propynyl]-N-hydroxyurea over 12 weeks.

FIG. 3 is a bar graph showing percent change in high sensitivity C reactive protein (hsCRP) in the presence of increasing doses of N-[3-[5-[(4-fluorophenyl)methyl]-2-thienyl]-1-methyl-2-propynyl]-N-hydroxyurea (VIA-2291).

FIG. 4A is a bar graph showing the change in non-calcified volume in multidetector computed tomography (MDCT) images in patients without N-[3-[5-[(4-fluorophenyl)methyl]-2-thienyl]-1-methyl-2-propynyl]-N-hydroxyurea compared to an average of patients who received any dose of N-[3-[5-[(4-fluorophenyl)methyl]-2-thienyl]-1-methyl-2-propynyl]-N-hydroxyurea.

FIG. 4B is a bar graph showing the percent of new plaque lesions in multidetector computed tomography (MDCT) images in patients without N-[3-[5-[(4-fluorophenyl)methyl]-2-thienyl]-1-methyl-2-propynyl]-N-hydroxyurea compared to an average of patients who received any dose of N-[3-[5-[(4-fluorophenyl)methyl]-2-thienyl]-1-methyl-2-propynyl]-N-hydroxyurea.

FIG. 5A is a line graph showing LTB4 production in patients who received 100 mg of N-[3-[5-[(4-fluorophenyl)methyl]-2-thienyl]-1-methyl-2-propynyl]-N-hydroxyurea (VIA-2291) compared to placebo.

FIG. 5B is a bar graph showing the percent change from baseline of LTE4 in patients who received 100 mg of N-[3-[5-[(4-fluorophenyl)methyl]-2-thienyl]-1-methyl-2-propynyl]-N-hydroxyurea (VIA-2291) compared to placebo.

FIG. 5C is a bar graph showing the percent change from baseline of hsCRP in patients who received 100 mg of N-[3-[5-[(4-fluorophenyl)methyl]-2-thienyl]-1-methyl-2-propynyl]-N-hydroxyurea (VIA-2291) compared to placebo.

FIG. 6A-D are photographs showing non-rupture prone and rupture prone plaques in patients who received 100 mg of N-[3-[5-[(4-fluorophenyl)methyl]-2-thienyl]-1-methyl-2-propynyl]-N-hydroxyurea (VIA-2291) compared to patients who received placebo.

FIG. 6E is a bar graph showing the ratio of necrotic core thickness and plaque thickness in non-rupture prone and rupture prone plaques in patients who received 100 mg of N-[3-[5-[(4-fluorophenyl)methyl]-2-thienyl]-1-methyl-2-propynyl]-N-hydroxyurea (VIA-2291) compared to patients who received placebo.

FIGS. 7A and B are bar graphs showing the fold change in expression of various proteins in non-rupture prone and rupture prone plaques in patients who received 100 mg of N-[3-[5-[(4-fluorophenyl)methyl]-2-thienyl]-1-methyl-2-propynyl]-N-hydroxyurea (VIA-2291) compared to patients who received placebo.

### SUMMARY OF INVENTION

In accordance with this invention, it has been found that the administration to patients of N-[3-[5-[(4-fluorophenyl)methyl]-2-thienyl]-1-methyl-2-propynyl]-N-hydroxyurea or pharmaceutically effective salts thereof is effective in preventing or treating atherosclerotic plaque, cardiovascular diseases, and other inflammatory diseases including chronic obstructive pulmonary disease (COPD), ocular inflammatory diseases, asthma, allergic rhinitis, rheumatoid arthritis, psoriasis, adult respiratory distress syndrome, inflammatory bowel disease, endotoxin shock syndrome, ischemia induced myocardial injury, and central nervous system pathology resulting from formation of leukotrienes following stroke or subarachnoid hemorrhage. In this manner the composition of the invention, N-[3-[5-[(4-fluorophenyl)methyl]-2-thienyl]-1-methyl-2-propynyl]-N-hydroxyurea, and its pharmaceutically acceptable salts are effective in treating and preventing various pathologies wherein the composition of the invention comprises less than 2% of the S-enantiomer.

The invention provides a composition comprising N-[3-[5-[(4-fluorophenyl)methyl]-2-thienyl]-1-methyl-2-propynyl]-N-hydroxyurea or pharmaceutically effective salts thereof wherein the compound comprises less than 2% of the S-enantiomer of N-[3-[5-[(4-fluorophenyl)methyl]-2-thienyl]-1-methyl-2-propynyl]-N-hydroxyurea. In one embodiment, the compound comprises less than 1% of the S-enantiomer of N-[3-[5-[(4-fluorophenyl)methyl]-2-thienyl]-1-methyl-2-propynyl]-N-hydroxyurea. In another embodiment, the composition is provided in a unit dosage form for oral administration wherein the composition is present in an amount of about 100 mg. In one aspect of this embodiment, said unit oral dosage form is a tablet or capsule.

The invention also provides a method of treating a leukotriene related pathology in a subject in need thereof comprising administering a composition comprising N-[3-[5-[(4-fluorophenyl)methyl]-2-thienyl]-1-methyl-2-propynyl]-N-hydroxyurea or pharmaceutically effective salts thereof wherein the compound comprises less than 2% of the S-enantiomer of N-[3-[5-[(4-fluorophenyl)methyl]-2-thienyl]-1-methyl-2-propynyl]-N-hydroxyurea.

### DETAILED DESCRIPTION

In accordance with this invention, it has been discovered that the administration to patients of N-[3-[5-[(4-fluorophenyl)methyl]-2-thienyl]-1-methyl-2-propynyl]-N-hydroxyurea, its pharmaceutically acceptable salts, or its pharmaceutically acceptable hydrolyzable esters is effective in treating patients susceptible to heart attack, stroke or peripheral arterial disease caused by atherosclerotic plaque atherosclerotic plaque, cardiovascular diseases, and other inflammatory diseases including chronic obstructive pulmonary disease (COPD), ocular inflammatory diseases, asthma, allergic rhinitis, rheumatoid arthritis, psoriasis, adult respiratory distress syndrome, inflammatory bowel disease, endotoxin shock syndrome, cancers including leukemias and lymphomas, ischemia induced myocardial injury, and central nervous system pathology resulting from formation of leukotrienes following stroke or subarachnoid hemorrhage.

In addition the administration of the composition of the invention or one or more of its pharmaceutically acceptable salts to patients are effective in the treatment of allergic diseases, such as asthma, allergic rhinitis, rhinosinusitis, atopic dermatitis and urticaria; fibrotic diseases such as airway remodeling in asthma, bronchiolitis obliterans after lung transplantation, idiopathic pulmonary fibrosis, scleroderma and asbestosis; other pulmonary syndromes such as acute lung injury or adult respiratory distress syndrome, viral bronchiolitis, obstructive sleep apnea, chronic obstructive pulmonary disease, cystic fibrosis and other forms of bronchiectasis and bronchopulmonary dysplasia; inflammatory diseases such as arthritis (including osteoarthritis and gout), glomerulonephritis, interstitial cystitis, psoriasis and inflammatory bowel disease; systemic inflammatory diseases such as rheumatoid arthritis, vasculitides (e.g. systemic lupus erythematosus, Churg-Strauss syndrome, and Henoch-Schonlein purpura) and transplant rejection; and cancer such as solid tumors (including melanoma, mesothelioma, pancreatic, lung, esophageal, prostate and colon cancers), leukemias and lymphomas.

The term “patient” includes any human or mammal subject who is susceptible to one or more diseases that are treatable or preventable using the composition of the invention and/or one or more of its pharmaceutically acceptable salts. This includes patients who in view of their family history, genetic testing or the presence of other risk factors (e.g., smoking, hypertension, high cholesterol, diabetes, obesity) have a predisposition to a disease that the composition of the invention and/or one or more of its pharmaceutically acceptable salts is effective in treating. Where the composition of the invention and/or one or more of its pharmaceutically acceptable salts is used in patients who are otherwise susceptible to a disease that the composition of the invention is effective in treating, which have not been diagnosed as having any of these diseases, the composition of the invention is used as a prophylaxis for these diseases. This means that the administration of the composition of the invention and/or one or more of its pharmaceutically acceptable salts reduces the likelihood of the onset of any one or more of these diseases.

In accordance with this invention, it is discovered that when the composition of the invention or one or more of its pharmaceutically acceptable salts are administered to patients, the composition exhibits its effect and minimizes or eliminates the toxicity or adverse effects commonly associated with certain N-hydroxyureas. This allows the composition of the invention or one or more of its pharmaceutically acceptable salts to be administered to human patients even at high dosages without producing the toxicity or degree of toxicity and concomitant level of adverse effects associated with certain N-hydroxyureas.

The term “halogen” includes all halogens, particularly, bromine, chlorine, fluorine and iodine.

By “pharmaceutically acceptable salt” it is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hem isulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The composition of the invention or one or more of its pharmaceutically acceptable salts which are used in accordance with the present invention exhibit stereoisomerism by virtue of the presence of one or more asymmetric or chiral centers in the composition. The present invention contemplates the various stereoisomers and mixtures thereof. Desired enantiomers are obtained by chiral synthesis from commercially available chiral starting materials by methods well known in the art, or may be obtained from mixtures of the enantiomers by resolution using known techniques.

According to certain embodiments of the invention, substantially all of the composition of the invention that is produced is the R-enantiomer. Only a small amount of S-enantiomer is present. This is advantageous because the S-enantiomer of the composition of the invention is often less therapeutically effective than the R-enantiomer and in some cases is toxic when administered to some patients. In specific embodiments, the composition of the invention produced has less than 5% of the S-enantiomer present by weight. In other specific embodiments, the composition of the invention produced has less than 4, 3, 2 or 1% of the S-enantiomer present by weight. In a preferred embodiment, the composition of the invention has less than 2% of the S-enantiomer present by weight. In a more preferred embodiment, the composition of the invention has less than 1% of the S-enantiomer present by weight.

In preventing and treating disease in patients by administering the composition of the invention and/or one or more of its pharmaceutically acceptable salts can be administered systemically either by injection, orally, or topically. In general the composition of the invention and/or one or more of its pharmaceutically acceptable salts can be administered to a human patient in any amount which is effective in preventing and treating disease in such patients. In carrying out such treatment and prevention, the composition of the invention and/or one or more of their pharmaceutically acceptable salts are preferably administered orally at a dosage of from about 25 to about 150 mg per day. In other more specific embodiments, the composition of the invention and/or one or more of their pharmaceutically acceptable salts is administered at a dosage of from about 50 to about 125 mg per day, from about 75 to about 100 mg per day or from about 100 to about 150 mg per day.

For treatment of certain severe life threatening diseases a higher dose of the composition of the invention and/or one or more of its pharmaceutically acceptable salts is contemplated. In certain embodiments, for the treatment of severe life threatening diseases, a dose of between about 0.3 and 3.0 mg/kg is administered. In other embodiments, for the treatment of severe life threatening diseases, a dose of up to 200 mg per day is administered. In certain specific embodiments, the composition of the invention and/or one or more of its pharmaceutically acceptable salts is administered in two 100 mg doses per day. According to specific embodiments, severe life threatening diseases include cancers including leukemias and lymphomas, adult respiratory distress syndrome and endotoxin shock syndrome.

In another embodiment, the composition of the invention and/or one or more of their pharmaceutically acceptable salts is administered at a dosage from about 0.2 to about 2.0 mg/kg of body weight of the patient per day when the composition of the invention is administered to children.

The dosages can be administered orally in solid oral unit dosage forms such as capsules, tablets, dragees, pills, powders, granules and the like, as well as liquid oral dosage forms such as solutions, syrups, suspensions, elixirs and the like. In general, the unit dosage form should contain the composition of the invention or its pharmaceutically acceptable salts in amounts of from about 25 to 150 mg. Of the unit oral dosage forms, capsules and tablets are especially preferred. When the drug is administered orally, it is generally administered at regular intervals conveniently at meal times or once daily.

The composition of the invention and/or its one or more of pharmaceutically acceptable salts is orally administered when used for treating diagnosed cardiovascular disease.

The composition of the invention and/or its one or more of pharmaceutically acceptable salts can be parenterally administered. The term “parenteral administration” refers to modes of administration which include intravenous, ocular, intraocular, intramuscular, intraperitoneal, subcutaneous and intra articular injection and infusion. Pharmaceutical compositions for parenteral administration comprise pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and non aqueous carriers, diluents, solvents or vehicles includes water, ethanol, polyols such as glycerol, propylene glycol, polyethylene glycol and the like and suitable mixtures thereof, vegetable oils, such as olive oil, and injectable organic esters such as ethyol oleate.

In a preferred embodiment, the composition of the invention and/or one or more of its pharmaceutically acceptable salts are administered ocularly when they are administered for the treatment of inflammatory eye disorders. In a more preferred embodiment, the composition of the invention and/or one or more of its pharmaceutically acceptable salts are administered intraocularly when they are administered for the treatment of inflammatory eye disorders.

The parenteral administration the composition of the invention and/or one or more of its pharmaceutically acceptable salts can be administered at the same daily dosage as that for oral administration, as explained above.

The dosage, in the case for systemic administration, varies in accordance with the requirement of the individual patient as determined by the treating physician. In general, however, the same daily dosage as that for oral administration, as explained above, is preferred, regardless of the method of administration of the systemic dose. The dosage can be administered as a single dosage or in several divided dosages proportionate with the dosage plan as determined by a physician in accordance with the requirements of the patient. In preparing the compositions for such systemic administration these compositions contain the composition of the invention and/or one or more of its pharmaceutically acceptable salts and a pharmaceutically acceptable carrier compatible with said composition or its salt. In preparing such compositions, any conventional pharmaceutically acceptable carrier can be utilized. In certain specific embodiments of the invention, the dosage is an oral dosage form. In specific embodiments, the oral dosage form contains 25, 50, 75 or 100 mg of the composition of the invention. According to a preferred embodiment, the oral dosage form contains about 80, 85, 90, 95, 100, 105, 110, 115, 120, 125, 130, 135, 140, 145 or 150 mg of the composition of the invention.

As pointed out, solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active composition can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

### EXAMPLES

### Example 1

### Phase 2 Acute Coronary Syndrome (ACS) Study

This study demonstrated the efficacy of treatment with N-[3-[5-[(4-fluorophenyl)methyl]-2-thienyl]-1-methyl-2-propynyl]-N-hydroxyurea in reducing leukotriene production at 12 weeks after an ACS event in patients and provided supporting imaging data evidence that such a reduction in leukotriene production may influence atherosclerosis. In this randomized, placebo-controlled study, 191 patients were randomized 3 weeks after an acute coronary syndrome (ACS) to receive 25, 50, or 100 mg N-[3-[5-[(4-fluorophenyl)methyl]-2-thienyl]-1-methyl-2-propynyl]-N-hydroxyurea or placebo qd for 12 weeks. Baseline assessments were performed at the start of treatment and these baseline results were compared with repeat assessments during various follow-up periods during the treatment study. A subset of 93 patients who had undergone a Multidetector (64 slice coronary) Computerized Tomography (MDCT) examination at baseline continued on study medication for a total of 24 weeks and underwent a repeat scan.

Patients received a single daily oral dose of 25 mg, 50 mg, or 100 mg of N-[3-[5-[(4-fluorophenyl)methyl]-2-thienyl]-1-methyl-2-propynyl]-N-hydroxyurea or matching placebo by administering 2 capsules as prepared in Example 3 for 12 weeks or 24 weeks.

Blood samples for measurement of ex vivo leukotriene B4 (LTB4) and high sensitivity C reactive protein (hsCRP), and urine samples for measurement of urinary leukotriene E4 (LTE4) levels were collected pre-dose on weeks 2, 6 and 12. Blood samples were assayed for ex vivo LTB4 by enzyme-linked immunosorbent assay, and for hsCRP by an immunoturbidimetric method. Urine samples were assayed for LTE4 using Liquid Chromatography with Tandem Mass Spectrometry (LC/MS/MS).

For those patients who continued on study medication for a total of 24 weeks, contrast-enhanced CT examination was performed at baseline and after 24 weeks of treatment with a 64-slice scanner (GE LightSpeed VCT; GE Healthcare, USA). Target plaque lesions were defined prospectively as non-calcified plaque with measurable low-density components of <60 HU situated in the proximal or middle portion of either the left main, left anterior ascending, left circumflex or right coronary artery causing at least 20% luminal stenosis. Prior to analysis of results, patients had their MDCT examinations evaluated twice by the same reviewer and also by a second reviewer for evaluation of intraobserver and interobserver variability of measurements.

### Results

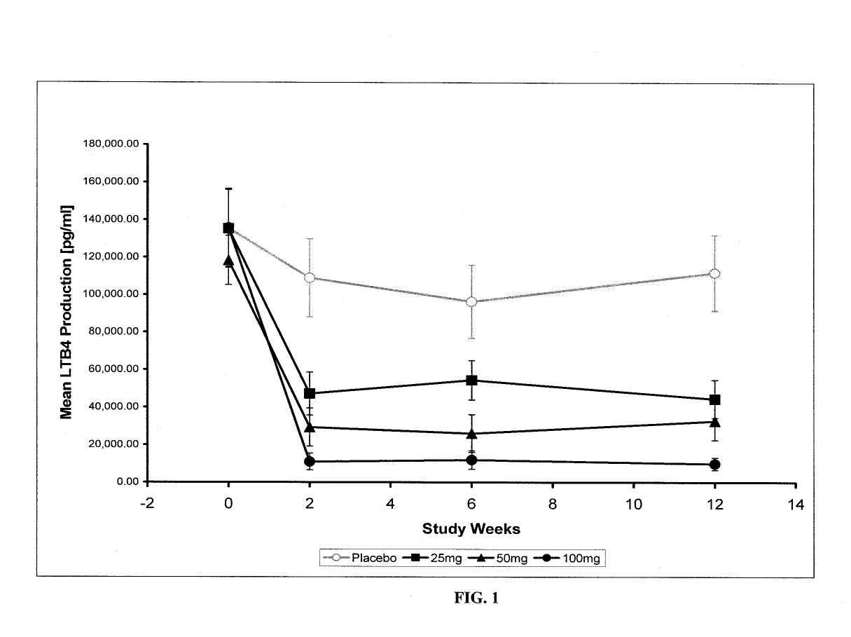

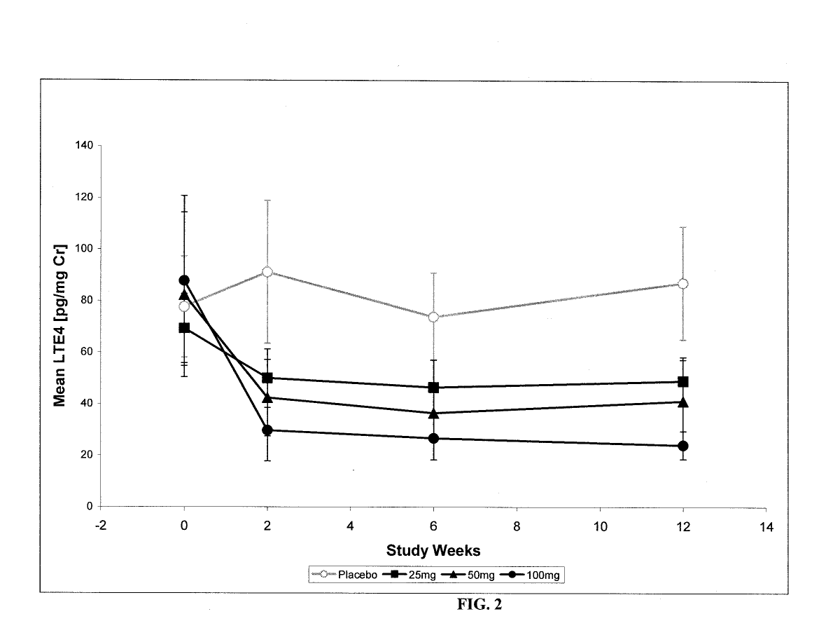

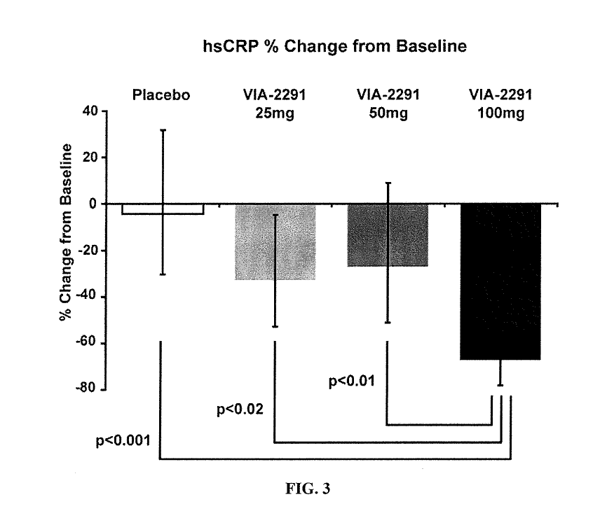

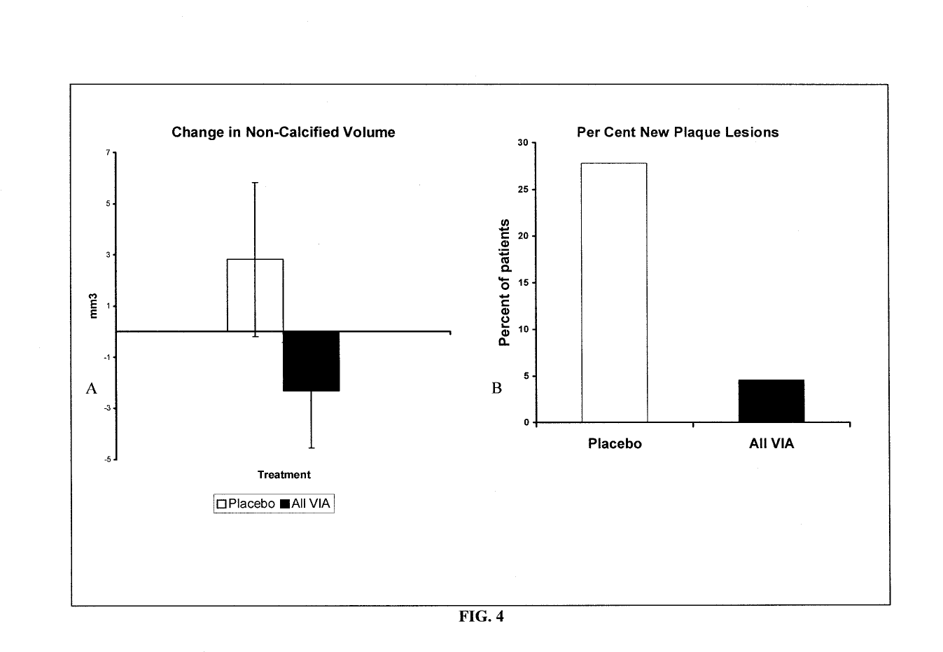

As shown in FIG. 1, N-[3-[5-[(4-fluorophenyl)methyl]-2-thienyl]-1-methyl-2-propynyl]-N-hydroxyurea significantly reduced ex vivo leukotriene LTB4 at trough drug levels at all doses (P<0.0001) and in a dose-dependent fashion, with approximately 80% inhibition in >90% of patients in the 100-mg group. N-[3-[5-[(4-fluorophenyl)methyl]-2-thienyl]-1-methyl-2-propynyl]-N-hydroxyurea also significantly reduced urinary leukotriene LTE4 at all doses, as shown in FIG. 2. HsCRP levels differed at baseline but decreased to 12 weeks for all doses of N-[3-[5-[(4-fluorophenyl)methyl]-2-thienyl]-1-methyl-2-propynyl]-N-hydroxyurea (Table 1). In a pre-specified assessment, there was a 67% decrease in hsCRP in the 100-mg group at 24 weeks, compared with placebo (P=0.0002, Table 1 and FIG. 3). There was a significant reduction in hsCRP within the N-[3-[5-[(4-fluorophenyl)methyl]-2-thienyl]-1-methyl-2-propynyl]-N-hydroxyurea 100 mg group between 12 and 24 weeks (P<0.01), and a significant increase in hsCRP within the placebo group during the same period (P<0.02); the 100 mg group was also significantly different from the 25 and 50 mg treatment groups in reducing hsCRP at 24 weeks. As shown in FIG. 4, all three doses of N-[3-[5-[(4-fluorophenyl)methyl]-2-thienyl]-1-methyl-2-propynyl]-N-hydroxyurea (VIA-2291) reduced non-calcified plaque volume and new plaque lesions as compared with placebo in serial MDCT images at 24 weeks.

TABLE 1

| | | | VIA-2291 | VIA-2291 | VIA-2291 |
|---|---|---|---|---|---|
| 12-week main | | Placebo | 25 mg | 50 mg | 100 mg |
| population | Statistics | (n = 48) | (n = 44) | (n = 38) | (n = 38) |
| Baseline | Median (25-75) | 1.1 (0.4, 4.0) | 1.5 (0.9, 2.5) | 2.0 (0.5, 4.7) | 0.7 (0.5, 2.6) |
| 12 weeks | Median (25-75) | 0.7 (0.3, 2.0) | 1.1 (0.6, 2.5) | 1.3 (0.4, 2.7) | 0.6 (0.3, 2.5) |
| Change from | Median (25-75) | −0.2 (−0.9, 0.1) | −0.2 (−1.1, 0.4) | −0.1 (−1.9, 0.1) | −0.3 (−0.8, 0.1) |
| baseline* | | | | | |
| | LSMEANS | −36.97% | −25.32% | −29.13% | −38.99% |
| | geometric mean | | | | |
| | (%) | | | | |
| | P-value change | 0.0002 | 0.0213 | 0.0119 | 0.0004 |
| | from baseline | | | | |
| | within group | | | | |
| | P-value (adj) vs. | | 0.6558 | 0.8627 | 0.9962 |
| | placebo | | | | |
| 24-week CT | | | VIA-2291 | VIA-2291 | VIA-2291 |
| substudy | | Placebo | 25 mg | 50 mg | 100 mg |
| population | Statistics | (n = 27) | (n = 23) | (n = 20) | (n = 18) |
| Baseline | Median (25-75) | 1.2 (0.4, 4.6) | 1.9 (0.8, 3.3) | 2.0 (0.4, 7.7) | 0.9 (0.5, 4.1) |
| 12 weeks | Median (25-75) | 0.7 (0.3, 2.3) | 1.1 (0.6, 2.4) | 1.7 (0.4, 4.3) | 0.6 (0.4, 2.0) |
| 12 weeks | Median (25-75) | −0.1 (−0.7, 0.1) | −0.1 (−1.5, 0.8) | −0.1 (−5.0, 0.6) | −0.3 (−2.4, 0.1) |
| change from | LSMEANS | −35.82% | −24.82% | −22.71% | −39.05% |
| baseline* | geometric mean | | | | |
| | (%) | | | | |
| | P-value change | 0.0108 | 0.1185 | 0.1877 | 0.0178 |
| | from baseline | | | | |
| | within group | | | | |
| | P-value (adj) | | 0.8688 | 0.8230 | 0.9953 |
| | vs. placebo | | | | |
| 24 weeks | Median (25-75) | 1.6 (0.5, 3.3) | 1.2 (0.7, 2.1) | 1.5 (0.6, 2.6) | 0.3 (0.2, 0.9) |
| 24 weeks | Median (25-75) | 0.0 (−0.5, 1.2) | −0.4 (−1.2, 0.3) | −0.2 (−3.7, 0.2) | −0.4 (−3.9, −0.2) |
| change from | LSMEANS | −4.19% | −32.91% | −27.05% | −67.16% |
| baseline* | geometric mean | | | | |
| | (%) | | | | |
| | P-value change | 0.7896 | 0.0271 | 0.1217 | <.0001 |
| | from baseline | | | | |
| | within group | | | | |
| | P-value (adj) | | 0.3313 | 0.6060 | 0.0002 |
| | vs. placebo | | | | |

### Example 2

### Phase 2 Carotid Endarterectomy (CEA) Study

This study demonstrated the efficacy of treatment with N-[3-[5-[(4-fluorophenyl)methyl]-2-thienyl]-1-methyl-2-propynyl]-N-hydroxyurea in stabilizing cardiovascular disease and atherosclerotic plaque in male and female patients with carotid stenosis undergoing elective carotid endarterectomy (CEA) surgery. In this randomized, double blind, placebo-controlled study, 50 patients with significant carotid artery stenosis (60-90%) were treated once daily for 12 weeks with orally administered 100 mg N-[3-[5-[(4-fluorophenyl)methyl]-2-thienyl]-1-methyl-2-propynyl]-N-hydroxyurea or placebo prior to undergoing CEA, at which time endarterectomy tissue (plaque) was collected and stored for subsequent tissue analysis. Baseline assessments are performed at the start of treatment and these baseline results were compared with repeat assessments during various follow-up periods of treatment. The treatment was conducted for twelve weeks at which time these baseline assessments were performed and compared.

Patients received a total single daily oral dose of 100 mg of N-[3-[5-[(4-fluorophenyl)methyl]-2-thienyl]-1-methyl-2-propynyl]-N-hydroxyurea or matching placebo by administering 2 capsules as prepared in Example 3 for 12 weeks.

Blood samples for measurement of ex vivo LTB4 and hsCRP, and urine samples for measurement of urinary LTE4 levels were collected pre-dose on weeks 2, 6 and 12. Blood samples were assayed for ex vivo LTB4 by enzyme-linked immunosorbent assay, and for hsCRP by an immunoturbidimetric method. Urine samples were assayed for LTE4 using Liquid Chromatography with Tandem Mass Spectrometry (LC/MS/MS).

At the end of 12 weeks treatment with 100 mg of N-[3-[5-[(4-fluorophenyl)methyl]-2-thienyl]-1-methyl-2-propynyl]-N-hydroxyurea, patients underwent CEA, at which time endarterectomy tissue (plaque) was collected, fixed in 10% formalin and paraffin blocks, and stored for subsequent tissue analysis. Standard immunohistochemical methods were used to stain all plaque samples. Prior to analysis of plaque immunohistology results, plaques were classified according to morphology by accepted methods (Virmani R. et al. A comprehensive morphological classification scheme for atherosclerotic lesions. Arterioscler Thromb Vasc Biol. 2000; 20:1262-1275). A portion of each of the plaques was also analyzed for inflammatory gene expression after isolation of total RNA and reverse transcription using a TaqMan® High Capacity cDNA assay.

### Analysis

LTB4, LTE4 and hsCRP biomarker data were assessed using change from baseline comparisons and an ANCOVA was used to compare the treatment groups where the covariate was the baseline value of the outcome measure. Model assumptions of normality and parallelism were checked, and as necessary, log transformations or tertile analyses were employed. All tests were performed two-sided with 0.05 level of significance with the exception of the gene expression analyses. Statistical analysis of gene-expression data was performed on delta Ct values by a two-sided t-test. Gene expression changes were considered meaningful with a fold-change of more than 2-fold in either direction or with a significance level of less than 0.1. For plaque endpoints a t-test was used to compare results between treatment groups.

### Results

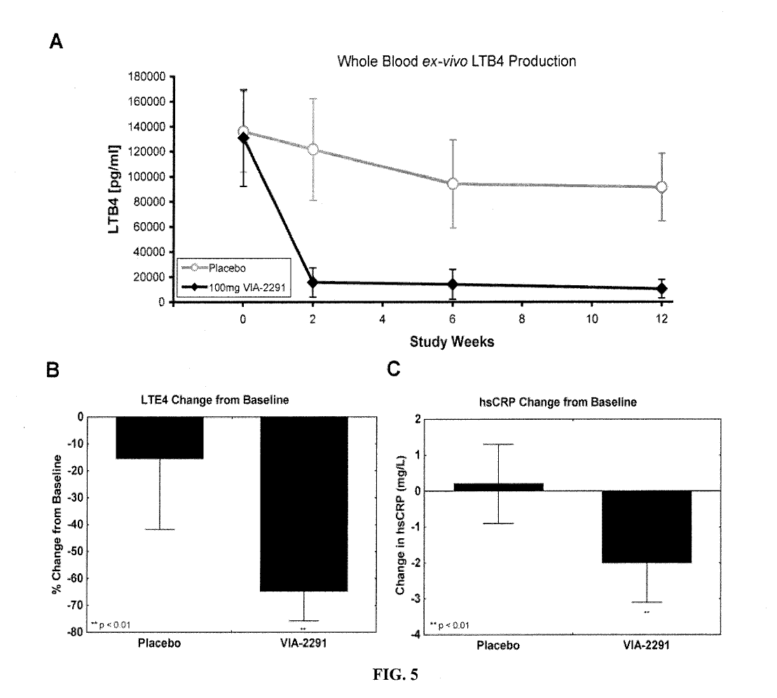

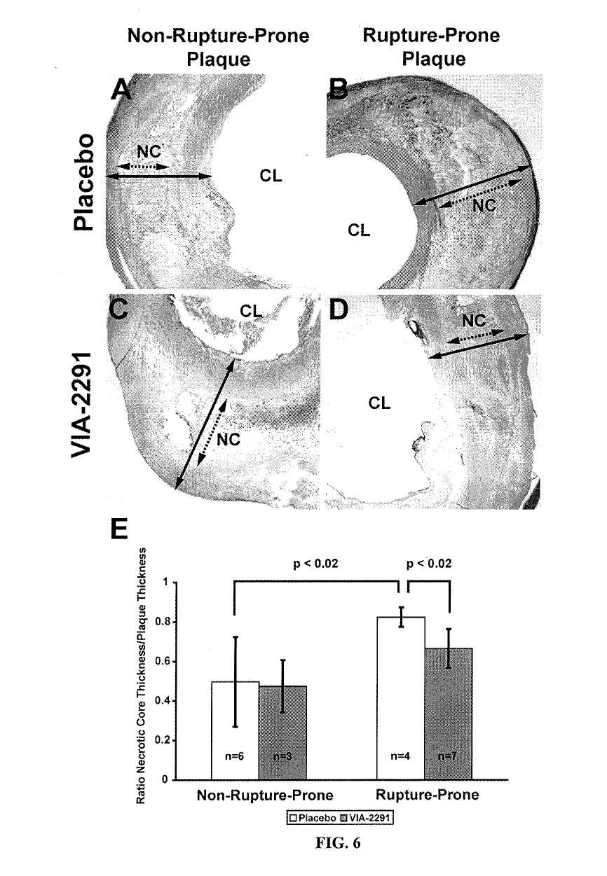

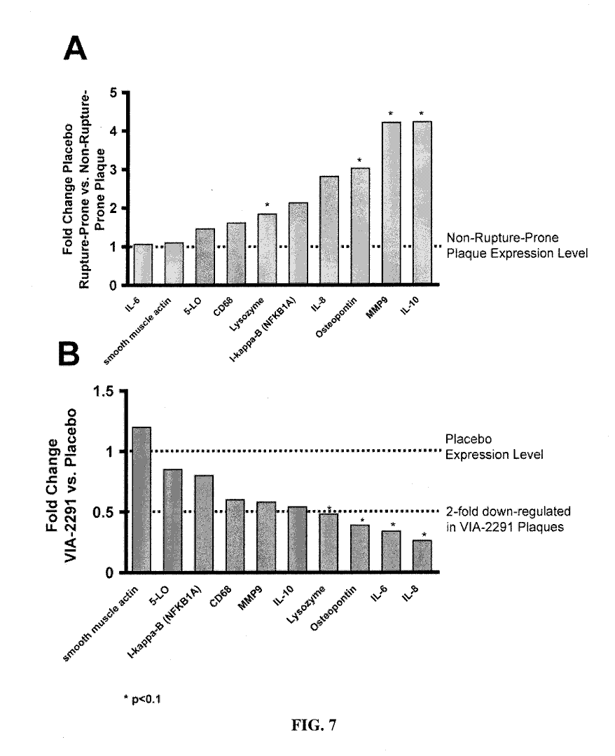

As shown in FIG. 5, compared with placebo, N-[3-[5-[(4-fluorophenyl)methyl]-2-thienyl]-1-methyl-2-propynyl]-N-hydroxyurea 100 mg statistically significantly reduced ex vivo LTB4 by approximately 90% (p<0.0001), urine LTE4 by 65% (p<0.01) and hsCRP by 2.0 mg/L (p<0.01) (secondary endpoints). An exploratory analysis of the relative necrotic core thickness in carotid plaques demonstrated that plaques with rupture-prone histological subtypes from patients treated with N-[3-[5-[(4-fluorophenyl)methyl]-2-thienyl]-1-methyl-2-propynyl]-N-hydroxyurea compared with placebo had significantly reduced (21%, p<0.02) relative necrotic core thickness (see FIG. 6). As shown in FIG. 7, these plaque subtypes also showed reduction in expression of inflammatory genes, including IL-6, IL 8, IL-10, MMP9, I-kappa-B, osteopontin in N-[3-[5-[(4-fluorophenyl)methyl]-2-thienyl]-1-methyl-2-propynyl]-N-hydroxyurea treated patients.

### Example 3

Capsules of N-[3-[5-[(4-fluorophenyl)methyl]-2-thienyl]-1-methyl-2-propynyl]-N-hydroxyurea were manufactured, by the following procedure.

N-[3-[5-[(4-fluorophenyl)methyl]-2-thienyl]-1-methyl-2-propynyl]-N-hydroxyurea capsules were manufactured in three strengths: 25 mg, 50 mg and 75 mg. These capsules were filled at three different fill weights of the 50% active formulation to achieve the three strengths. The ingredients and packaging components were identical for all three strengths.

N-[3-[5-[(4-fluorophenyl)methyl]-2-thienyl]-1-methyl-2-propynyl]-N-hydroxyurea capsules were manufactured using a common wet granulation made up of seven sub-batches, containing 50% N-[3-[5-[(4-fluorophenyl)methyl]-2-thienyl]-1-methyl-2-propynyl]-N-hydroxyurea, Lactose monohydrate, Pregelatinzed starch, Sodium Starch Glycolate, Povidone and USP water. The seven sub-batches were dried, milled and blended with crospovidone, glyceryl behenate, and magnesium stearate. The milled and blended material was then encapsulated to designated fill weight. The batch composition of the common granulation is shown on Table 2. The batch composition of N-[3-[5-[(4-fluorophenyl)methyl]-2-thienyl]-1-methyl-2-propynyl]-N-hydroxyurea Capsules, 25 mg is shown in Table 3, the batch composition of the N-[3-[5-[(4-fluorophenyl)methyl]-2-thienyl]-1-methyl-2-propynyl]-N-hydroxyurea Capsules, 50 mg is shown in Table 4 and the batch composition of N-[3-[5-[(4-fluorophenyl)methyl]-2-thienyl]-1-methyl-2-propynyl]-N-hydroxyurea Capsules, 75 mg is shown in Table 5. The invention also provides N-[3-[5-[(4-fluorophenyl)methyl]-2-thienyl]-1-methyl-2-propynyl]-N-hydroxyurea capsules containing 100 mg of N-[3-[5-[(4-fluorophenyl)methyl]-2-thienyl]-1-methyl-2-propynyl]-N-hydroxyurea.

TABLE 2
Batch Composition of Compound X Capsules, Common Granulation

| | Concentration | Theoretical Batch |
|---|---|---|
| Ingredient | (% w/w) | Quantity(g) |
| Common Granulation | | |
| (Sub-Batches A-G) | | |
| Compound X | 50.00 | 492.9 |
| Latose, Monohydrate, NF/EP | 24.00 | 236.6 |
| (Fastflo 316) | | |
| Pregelatinized Starch, NF/EP | 12.00 | 118.3 |
| (Starch 1500) | | |
| Sodium Starch Glycolate, NF/EP | 5.00 | 49.3 |
| Povidone, USP/EP (D29-32) | 3.00 | 29.6 |
| Purified Water, USP/EP | —* | 410.0* |
| Purified Water, USP/EP | —* | QS* |
| Blending Process for | | |
| Combined Sub-Batches A-G | | |
| Crospovidone (Kollidon (CL), | 2.00 | 138.0 |
| NF/EP | | |
| Glyceryl Behenate (Compritol 888 | 3.00 | 207.0 |
| ATO), NF/EP | | |
| Magnesium Stearate (NonBovine | 1.00 | 69.0 |
| HyQual R), NF/EP | | |
| Total | 100.0 | — |

*Water was removed by drying after wet granulation, not present in final dosage form

TABLE 3
Batch Composition of Compound X Capsules, 25 mg
Batch Size: 20,000 Capsules

| | Concentration | Capsule | Batch |
|---|---|---|---|
| Ingredient | (% w/w) | Quantity | Quantity (g) |
| Capsule Common | 50% | 50.0 mg | 1000.0 |
| Granulation | | | |
| Capsules, Hard Gelatin, | — | 20,000 each | 20,000 |
| Swedish Orange, Size #2 | | | capsules |

TABLE 4
Batch Composition of Compound X Capsules, 50 mg
Batch Size: 56,000 Capsules

| | Concentration | Capsule | Batch |
|---|---|---|---|
| Ingredient | (% w/w) | Quantity | Quantity (g) |
| Compound X Capsule | 50% | 100.0 mg | 5600.0 |
| Common Granulation | | | |
| Capsules, Hard Gelatin, | — | 56,000 each | 56,000 |
| Swedish Orange, Size #2 | | | capsules |

TABLE 5
Batch Composition of Compound X Capsules, 75 mg
Batch Size: 20,000 Capsules

| | Concentration | Capsule | Batch |
|---|---|---|---|
| Ingredient | (% w/w) | Quantity (mg) | Quantity (g) |
| Compound X Capsule | 50% | 150.0 mg | 3000 |
| Common Granulation | | | |
| Capsules, Hard Gelatin, | | 20,000 each | 20,000 |
| Swedish Orange, Size #2 | | | capsules |

What is claimed is:

1. A composition comprising N-[3-[5-[(4-fluorophenyl)methyl]-2-thienyl]-1-methyl-2-propynyl]-N-hydroxyurea or pharmaceutically effective salts thereof wherein the compound comprises less than 2% of the S-enantiomer of N-[3-[5-[(4-fluorophenyl)methyl]-2-thienyl]-1-methyl-2-propynyl]-N-hydroxyurea.

2. The composition of claim 1, wherein the composition comprises less than 1% of the S-enantiomer of N-[3-[5-[(4-fluorophenyl)methyl]-2-thienyl]-1-methyl-2-propynyl]-N-hydroxyurea.

3. The composition of claim 1, wherein the composition is provided in a unit dosage form for oral administration and wherein the composition is present in an amount of about 100 mg.

4. The composition of claim 3, wherein said unit oral dosage form is a tablet or capsule.

5. A method of treating a leukotriene related pathology in a subject in need thereof comprising administering the composition of claim 1 to the subject.
